(19) United States
(12) Patent Application Publication
(10) Pub. No.: US 2006/0083754 A1
(43) Pub. Date: Apr. 20, 2006

(54) Pharmaceutical combinations comprising corticoids and immunosuppressants for treating corticoid- and/or calcineurin inhibitors-resistant diseases

(76) Inventor: Anthony Winiski, Wien (AT)

Correspondence Address: NOVARTIS;CORPORATE INTELLECTUAL PROPERTY, ONE HEALTH PLAZA 104/3, EAST HANOVER, NJ, 07936-1080, US

(21) Appl. No.: 10/544,918

(22) PCT Filed: Feb. 9, 2004
(86) PCT No.: PCT/EP04/01179

§ 371 Date: Aug. 9, 2005

(30) Foreign Application Priority Data
Feb. 10, 2003 (GB) 03029915
Jul. 14, 2003 (GB) 03164365

(51) Int. Cl. A61K 39/385

(52) U.S. Cl. 424/193.1

(57) ABSTRACT

The use of a combination of a corticoid and a compound of formula I for the preparation of a medicament for the treatment of a corticoid-resistant disease and/or a calcineurin inhibitor-resistant disease, e.g. where T cells are involved in the pathophysiology.

The present invention relates to new uses of pharmaceutical combinations, e.g. combinations of pharmaceutically active compounds.

Substances that inhibit T-cell proliferation, i.e. proliferation of T lymphocytes, have been used therapeutically as immunosuppressive and/or immunomodulatory agents, e.g. including corticoids, such as glucocorticoids, and derivatives thereof. However, it is known that resistance to such agents, e.g. corticoid-resistance, exists or may develop, which means that such agents are then not active, or not sufficiently active, for the successful treatment of a variety of diseases.

Surprisingly, we have now found that resistance to such agents may be overcome, if a combination treatment is applied.

In one aspect the present invention provides the use of a combination of a corticoid and a compound of formula

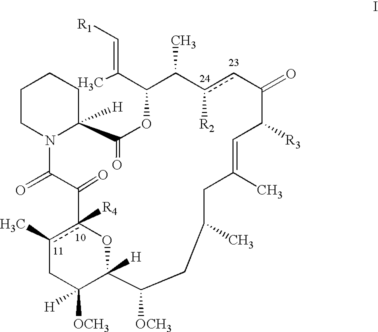

wherein either

R₁ is a group (a) of formula

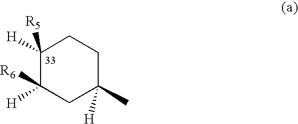

wherein

R₅ is chloro, bromo, iodo or azido,

R₆ is hydroxy or methoxy, and

R₄ is hydroxy and there is a single bond in 10,11 position; or absent, and there is a double bond in 10,11 position or

R₁ is a group (b) or (c) of formula

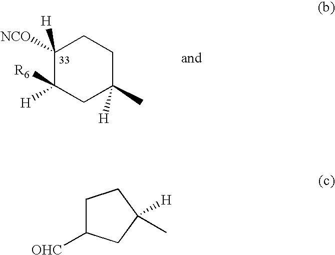

wherein

R₆ is as defined above, and

R₄ is hydroxy and there is a single bond in 10,11 position,

R₂ is oxo and there is a single bond in 23,24 position; hydroxy and there is a single or double bond in 23,24 position; or absent and there is a double bond in 23,24 position;

R₃ is methyl, ethyl, propyl or allyl,

for the manufacture of a medicament for the treatment of a corticoid-resistant disease and/or a calcineurin inhibitor-resistant disease, e.g. for the treatment of a disease wherein a compound of formula I alone or a corticoid alone is ineffective or insufficiently effective.

Therapeutically ineffective or insufficiently effective means that a compound of a combination of the present invention alone does not show efficacy or does not show sufficient efficacy in a clinical environment. We have established in vitro systems, e.g. as described herein, in which a corticoid alone or a calcineurin inhibitor alone, under certain conditions, does not inhibit T-cell proliferation to a degree necessary for therapeutic treatment. These systems thus serve as an in vitro experimental models of corticoid and/or calcineurin inhibitor resistance.

We have defined that a compound of the combination of the present invention alone with an inhibitory effect of less than 35%, e.g. less than 25%, such as less than 20%, is insufficient for therapeutic treatment. On the other hand, we have defined that a compound of a combination of the present invention alone is sufficient for therapeutic treatment if an inhibitory effect of at least 60% and more, such as 80%, 90%, up to practically 100% is achieved.

Compounds of formula I are e.g. disclosed in EP-B-0427680, wherein in a compound of formula I preferred substituents are indicated, which preferred substituents are also preferred substituents in the present application; e.g. in a compound of formula I each single defined substituent may be a preferred substituent, e.g. independently of each other substituent defined; e.g. including a compound of formula I_{P}, such as disclosed in EP-B-0427680 in Example 66a as “33-epi-33-chloro-FR 520”, also known as “ASM981”.

In another aspect the present invention provides the use of a combination of a corticoid and a compound of formula I according to the present invention, wherein a compound of formula I is a compound of formula

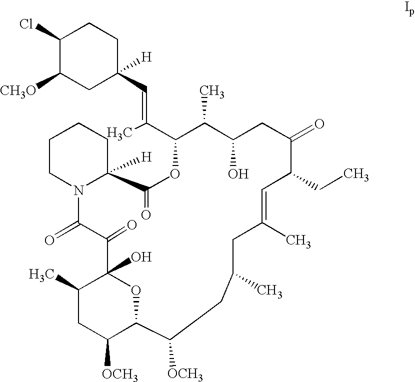

A corticoid in a combination of the present invention includes pharmaceutically active corticoids and derivatives thereof, e.g. including corticosteroids, such as glucocorticoids (i.e. having glucocorticoid-like activity), e.g. which show pharmaceutical activity, as well as nonsteroidal ligands of the glucocorticoid receptor,

e.g. including corticoids in free form and in the form of
esters, e.g. including mono- and diesters, e.g. in the form of salts, e.g. sodium,
acetals and ketals, such as acetonides,

e.g. in the form of salts and solvates, where applicable.

Examples include alclomethasone, (e.g. -diproprionate), amicinonide, beclomethasone (e.g. -dipropionate), betamethasone (e.g. -acetate, -benzoate, -dipropionate, sodium phosphate, -valerate), budesonide, carbenoxolone (e.g. -sodium), ciclesonide, clobetasole (e.g. propionate), clobetasone (e.g. butyrate), clocortolone (e.g. -acetate, -pivalate), cloprednol, corticosterone, corticotropin (e.g. -zinc hydroxide), cortisol, cortisone (e.g. -acetate), cortivazol, deflazacort, descinolone (e.g. -acetonide), desonide, dexamethasone (e.g. sodium phosphate, -acetate, -isomicotinate), desoxymethasone, diflorasone (e.g. diacetate), difluocortolone (e.g. -pivalate, valerate), difluprednate, flucloronide, fludrocortisone, fludroxycortide, flumethasone (e.g. -pivalate), flunisolide, fluocortin (butyl), fluocinonide, fluocinolone (e.g. -acetonide), fluocortolone (e.g. -caproate), fluorometholone, fluperolone (e.g. -acetate), fluprednidene (e.g. -21-acetal, -acetate), fluprednisolone (e.g. -valerate), flurandrenolide, fluticasone (e.g. -propionate, valerate), formocortal, halcinonide, halobetasol (e.g. -propionate), halomethasone (e.g. monohydrate), hydrocortisone (e.g. -acetate, -buteprat, -butyrate, cypionate, -sodium phosphate, -sodium succinate, -hemisuccinate, -valerate), medrysone, methylprednisolone (e.g. -acetate, -sodium phosphate, -sodium succinate, aceponate), momethasone (e.g. fuorate), nivazol, paramethasone (e.g. -acetate), prednicarbate, prednisolone (e.g. including -acetate, -hemisuccinate, -sodium phosphate, -sodium succinate, -tebutate), prednisone, prednisolone, prednival, prednylidene, rofleponide (e.g. palmitate), ticabesone (e.g. -propionate), tipredane, tralonide, triamcinolone (e.g. -acetonide, -acetonide sodium phosphate, -diacetate), e.g., and pharmacodynamic equivalents thereof, preferably hydrocortisone, betamethasone, e.g. betamethasone 17-valerate, or dexamethasone.

Pharmacodynamic equivalents are meant to include corticoids, having similar pharmaceutical activity in comparison with specific corticoids listed herein.

Pharmaceutical excipient includes e.g. appropriate carrier and/or diluent, e.g. including fillers, binders, disintegrators, flow conditioners, lubricants, sugars and sweeteners, fragrances, preservatives, stabilizers, wetting agents and/or emulsifiers, solubilizers, salts for regulating osmotic pressure and/or buffers.

In another aspect the present invention provides the use of a combination of a corticoid and a compound of formula I according to the present invention, wherein the corticoid is selected from the group consisting of hydrocortisone, betamethasone, e.g. betamethasone 17-valerate, and dexamethasone.

A compound of a combination according to the present invention may be in free form, in the form of a salt, in solvate form or in the form of a salt and a solvate, where salts and/or solvates exist.

In another aspect the present invention provides
the use of a combination of a corticoid and a compound of formula I according to the present invention, wherein a compound of formula I is in the form of a salt;
the use of a combination of a corticoid and a compound of formula I according to the present invention, wherein the corticoid is in the form of a salt;
the use of a combination of a corticoid and a compound of formula I according to the present invention, wherein a compound of formula I and a corticoid both are in the form of a salt.

Corticoid-resistant diseases are known and include, e.g.
alopecia, e.g. alopecia totalis or alopecia universalis,
allergies, e.g. contact allergies,
amyloidosis, e.g. systemic amyloidosis,
arteritis, e.g. Takayasu's arteritis,
arthritis, e.g. (juvenile) rheumatoid arthritis, juvenile oligoarthritis, sarcoidosis arthritis,
arthropathy, e.g. spondyloarthropathy,
asthma, e.g. bronchial asthma, chronic asthma,
colitis,
conjunctivitis, e.g. keratoconjunctivitis,
Crohn's disease, including corticosteroid-resistant pyoderma gangrenosum associated with Crohn's disease, refractory Crohn's disease,
cystic fibrosis,
dermatitis (=dermatosis), such as contact, atopic, allergic contact and solar dermatitis, lichenoid dermatitis, ulcerative dermatitis,
Multiple Sclerosis, e.g. Encephalo Multiple Sclerosis,
eczema,
Graves diseases, e.g. Graves' ophthalmopathy,
Graft Versus Host Disease (GVHD),
hemangiomas,
Hepatitis,
Inflammatory Bowel Disease (IBD),
insulin-dependent diabetes,
intraocular inflammatory diseases,
keratitis,
Macrophage activation syndrome,
myasthenia, e.g. myasthenia gravis,
myelitis, e.g. encephalomyelitis,
myositis, e.g. (juvenile) dermatomyositis (DM), polymyositis (PM) and inclusion body myositis (IBM),
nephritis, e.g. glomerulonephritis, nephritic syndrome,
ophthalmia, e.g. sympathetic ophthalmia,
pneumonitis,
polyarthritis, e.g. chronic polyarthrits such as Still's disease,
psoriasis,
pulmonary or lung diseases, e.g. chronic obstructive pulmonary disease (COPD),
retinal detachment
sarcoidosis or neurosarcoidosis,
scleritis,
sclerosis, e.g. glomerulosclerosis,
septic shock,
Sjogren's syndrome,
systemic lupus erythematosus,
transplant rejection, e.g. (renal or kidney) allograft rejection,
thrombocytopenic purpura, e.g. immune thrombocytopenic purpura (ITP), (chronic) idiopathic thrombocytopenic purpura,
ulcerative colitis,
urticaria, e.g. (chronic) idiopathic urticaria, urticarial vasculitis,
uveitis, such as anterior uveitis, chronic uveitis, peripheral uveitis, refractory uveitis, Behcet's uveitis, granulomatous uveitis such as Vogt-Koyanagi-Harada disease.

In another aspect the present invention provides the use of a combination of a corticoid and a compound of formula I, e.g. a compound of formula Iₚ, according to the present invention, wherein the disease is a disease in which T cells (i.e. T lymphocytes) are involved in the pathophysiology of the disease, such as T-cell mediated acute or chronic inflammatory diseases or disorders or autoimmune diseases,

e.g. a disease selected from the group consisting of
Graft Versus Host Disease (GVHD),
autoimmune diseases and inflammatory conditions, in particular inflammatory conditions with an etiology including an autoimmune component, such as hematological disorders, including e.g. hemolytic anemia, aplastic anemia, pure red cell anemia and idiopathic thrombocytopenia, rheumatoid arthritis, systemic Lupus erythematosus, polychondritis, scleroderma, Wegener's granulomatosis, chronic active hepatitis, Hashimoto's thyroiditis, Steven-Johnson syndrome, idiopathic sprue, autoimmune inflammatory bowel disease, Graves disease, sarcoidosis, multiple sclerosis, interstitial lung fibrosis, Myasthenia gravis, glomerulonephritis (with and without nephritic syndrome), juvenile dermatomyositis, juvenile diabetes (diabetes mellitus type I), immune-mediated conditions of the eye, e.g. uveitis (anterior and posterior), keratoplasty, chronic keratitis, keratoconjunctivitis sicca, vernal keratoconjunctivitis;
cutaneous manifestations of immunologically-mediated illnesses;
inflammatory and hyperproliferative skin diseases, such as psoriasis, atopic dermatitis, contact dermatitis and further eczematous dermatitides, seborrheic dermatitis, Lichen planus, Pemphigus, bullous Pemphigoid, Epidermolysis bullosa, urticaria, angioedemas, vasculitides, erythemas, cutaneous eosinophilias, Lupus erythematosus and acne;
allergic conditions, e.g. vernal conjunctivitis, ocular allergy;
inflammatory nervous injury, e.g. brain inflammation;
cerebral anoxia, hypoxia or ischemia;
asthma,
chronic obstructive pulmonary disease (COPD),
inflammatory bowel disease (IBD), including ulcerative colitis and Crohn's disease;
multi-drug resistance (MDR), and
Alopecia areata,

wherein a compound of formula I, e.g. a compound of formula Iₚ, alone or a corticoid alone is ineffective or insufficiently effective.

In another aspect the present invention provides the use of a combination of a corticoid and a compound of formula I according to the present invention, wherein the disease is selected from the group consisting of atopic dermatitis, psoriasis, psoriatic arthritis, rheumatoid arthritis, asthma, ulcerative colitis and Crohn's disease.

Treatment includes treatment and prophylaxis.

For such treatment, the appropriate dosage will, of course, vary depending upon, for example, the chemical nature and the pharmacokinetic data of a compound of the present invention employed, the individual host, the mode of administration and the nature and severity of the conditions being treated. However, in general, for satisfactory results in larger mammals, for example humans, a calcineurin inhibitor, e.g. pimecrolimus, may be provided as a solution or cream in the range from about 0.1% to 5% w/v or w/w when administered locally, wherein the dosage will depend on the kind of disease to be treated as well as on the administration site, or in the range of 10 mg to 120 mg per patient, e.g. 0.1 mg/kg to 2 mg/kg, of a calcineurin inhibitor, e.g. pimecrolimus, when administered systemically, e.g. orally, and the corticoid is given in dosages as known for standard therapies, such as e.g. in a range of 0.5 to 5% in case of topical application or in a range of 0.25 to 2500 mg, preferably 1 to 500 mg, such as 1 to 50 mg, when administered systemically, e.g. orally.

In another aspect the present invention provides the use of a combination of a corticoid and a calcineurin inhibitor for the manufacture of a medicament, e.g. a pharmaceutical composition, for the treatment of a corticoid-resistant disease and/or a calcineurin inhibitor-resistant disease wherein T cells are involved in the pathophysiology of the disease, with the proviso that focal segmental glomerulosclerosis wherein T cells are involved in the pathophysiology are excluded, e.g. for the treatment of diseases wherein a calcineurin inhibitor or a corticoid alone is ineffective or insufficiently effective.

In another aspect the present invention provides the use of a combination of a calcineurin inhibitor and a corticoid according to the present invention, wherein the disease is selected from the group consisting of
Graft-Versus-Host Diseases (GVHD),
autoimmune diseases and inflammatory conditions, in particular inflammatory conditions with an etiology including an autoimmune component, such as hematological disorders, including e.g. hemolytic anemia, aplastic anemia, pure red cell anemia and idiopathic thrombocytopenia, rheumatoid arthritis, systemic Lupus erythematosus, polychondritis, scleroderma, Wegener's granulomatosis, chronic active hepatitis, Hashimoto's thyroiditis, Steven-Johnson syndrome, idiopathic sprue, autoimmune inflammatory bowel disease, Graves disease, sarcoidosis, multiple sclerosis, interstitial lung fibrosis, Myasthenia gravis, glomerulonephritis (with and without nephritic syndrome), juvenile dermatomyositis, juvenile diabetes (diabetes mellitus type I), immune-mediated conditions of the eye, e.g. uveitis (anterior and posterior), keratoplasty, chronic keratits, keratoconjunctivitis sicca, vernal keratoconjunctivitis; cutaneous manifestations of immunologically-mediated illnesses;
inflammatory and hyperproliferative skin diseases, such as psoriasis, atopic dermatitis, contact dermatitis and further eczematous dermatitides, seborrheic dermatitis, Lichen planus, Pemphigus, bullous Pemphigoid, Epidermolysis bullosa, urticaria, angioedemas, vasculitides, erythemas, cutaneous eosinophilias, Lupus erythematosus and acne;
allergic conditions, e.g. vernal conjunctivitis, ocular allergy;
inflammatory nervous injury, e.g. brain inflammation;
cerebral anoxia, hypoxia or ischemia;
asthma,
inflammatory bowel disease (IBD), including ulcerative colitis and Crohn's disease;
multi-drug resistance (MDR),
ulcers, e.g. gastric ulcers
vascular damage caused by ischemic diseases and thrombosis
necrotizing lesions associated with thermal burns and
Alopecia areata,

wherein a calcineurin inhibitor alone or a corticoid alone is ineffective or insufficiently effective.

“Corticoid-resistant disease” and/or “calcineurin inhibitor-resistant disease” are as defined above.

Calcineurin is a calcium/calmodulin-regulated protein phosphatase involved in intracellular signalling. For reviews on calcineurin, see e.g. Rusnak and Mertz, Physiol. Rev. 80, 1483-1521 (2000) and Feske et al., Biochem. Biophys. Commun. 311, 1117-1132 (2003). Calcineurin inhibitors are substances which block calcineurin dephosphorylation of appropriate substrates.

A calcineurin inhibitor of the present invention is preferably an immunophilin binding compound having calcineurin inhibitory activity.

Immunophilin binding calcineurin inhibitors are compounds forming calcineurin inhibiting complexes with immunophilins, e.g. cyclophilin and macrophilin.

Examples of cyclophilin-binding calcineurin inhibitors are cyclosporins or cyclosporin derivatives (hereinafter cyclosporins) and examples of macrophilin-binding calcineurin inhibitors are ascomycin and ascomycin derivatives (hereinafter ascomycins), see e.g. Liu et al., Cell 66, 807-815 (1991) and Dumont et al., J. Exp. Med., 176, 751-780 (1992), as well as tacrolimus (FK506).

Cyclosporins and their preparation are e.g. disclosed in U.S. Pat. No. 4,117,118, wherein in a compound of formula I preferred substituents are indicated, which preferred substituents are also preferred substituents in the present application; e.g. in a compound of formula I each single defined substituent may be a preferred substituent, e.g. independently of each other substituent defined. Cyclosporin, originally extracted from the soil fungus Potypaciadium infilatum, has a cyclic 11-amino acid structure and includes e.g. Cyclosporins A through I, such as Cyclosporin A, B, C, D and G, preferably Cyclosporin A.

Ascomycins and their preparation are known. Ascomycin (FR 520) is a macrolide antibiotic disclosed e.g. in U.S. Pat. No. 3,244,592 and in EP 349061, wherein in a compound of formula I preferred substituents are indicated, which preferred substituents are also preferred substituents in the present application; e.g. in a compound of formula I each single defined substituent may be a preferred substituent, e.g. independently of each other substituent defined. A wide range of ascomycin derivatives are known, which are either naturally occurring amongst fungal species or are obtainable by manipulation of fermentation procedures or by chemical derivatization.

Ascomycins include e.g. a compound of formula I, as described above, preferably pimecrolimus.

In another aspect the present invention provides the use of a combination of a corticoid and a calcineurin inhibitor according to the present invention, wherein a calcineurin inhibitor is a compound of formula I, wherein the substituents are as described above, preferably a compound of formula I_{P}.

In another aspect the present invention provides the use of a combination of a calcineurin inhibitor and a corticoid according to the present invention, wherein a calcineurin inhibitor is a compound of formula

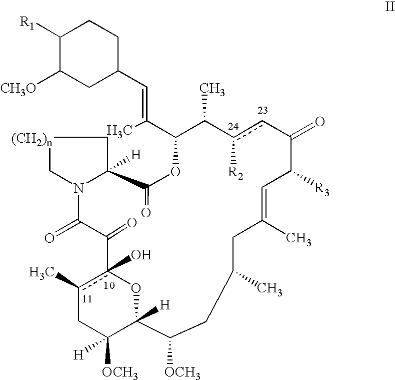

wherein

R₁ is hydroxy or protected hydroxy,

R₂ is hydrogen, hydroxyl or protected hydroxyl,

R₃ is methyl, ethyl, propyl or allyl,

n is an integer of 1 or 2, and

the symbol of a line and dotted line is a single bond.

Compounds of formula II are e.g. disclosed in EP-B-0184162, wherein in a compound of formula I preferred substituents are indicated, which preferred substituents are also preferred substituents in the present application; e.g. in a compound of formula I of the EP-B-0184162, each single defined substituent may be a preferred substituent, e.g. independently of each other substituent defined. A preferred compound is the compound FK 506 (tacrolimus) of formula II_{FK}.

In another aspect the present invention provides the use of a combination of a calcineurin inhibitor and a corticoid according to the present invention, wherein a calcineurin inhibitor is a compound of formula

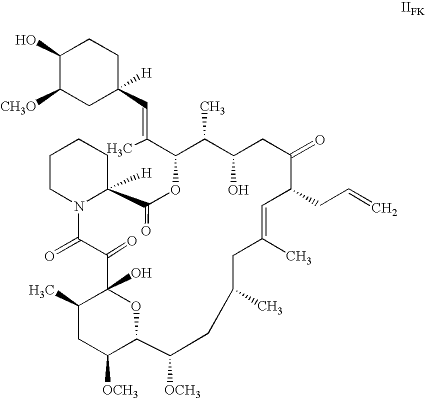

In another aspect the present invention provides the use of a combination of a calcineurin inhibitor and a corticoid according to the present invention, wherein a calcineurin inhibitor is a compound of formula

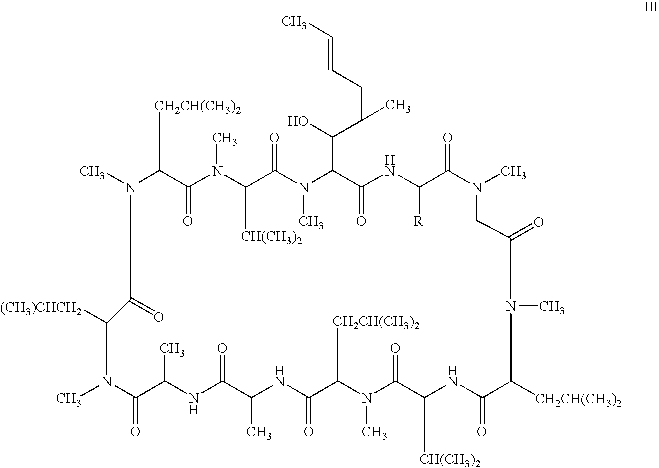

wherein R is methyl, ethyl, propyl, isopropyl or —CH(OH)CH₃, preferably R is ethyl (Cyclosporin A).

In another aspect the present invention provides the use of a combination of a corticoid and a calcineurin inhibitor according to the present invention, wherein a corticoid is selected from corticoids as described above, preferably the corticoid is selected from the group consisting of hydrocortisone, betamethasone, e.g. betamethasone 17-valerate, and dexamethasone.

A compound of a combination of a calcineurin inhibitor and a corticoid may be in free form, in the form of a salt, in solvate form or in the form of a salt and a solvate, where salts and/or solvates exist.

In another aspect the present invention provides
the use of a combination of a corticoid and a calcineurin inhibitor according to the present invention, wherein a calcineurin inhibitor is in the form of a salt;
the use of a combination of a corticoid and a calcineurin inhibitor according to the present invention, wherein the corticoid is in the form of a salt;
the use of a combination of a corticoid and a calcineurin inhibitor according to the present invention, wherein a calcineurin inhibitor and a corticoid both are in the form of a salt.

A combination according to the present invention may contain one or more calcineurin inhibitors and one or more corticoids, and contains preferably one calcineurin inhibitor and one corticoid.

The compounds of a combination of the present invention may be used, e.g. administered, in free form or in the form of a pharmaceutically acceptable salt, e.g. an acid addition salt or metal salt; optionally in the form of a solvate. Corticoids may additionally be in the form of esters, acetonides, e.g. and additionally in the form of salts. The compounds of a combination of the present invention in the form of a salt/ester/acetonide/solvate/ exhibit the same order of activity as the compounds used in the present invention in free form; optionally in the form of a solvate.

Treatment and dosage are as described above for a combination of a compound of formula I and a corticoid.

The ratio of calcineurin inhibitor, e.g. including a compound of formula I, II or III, to corticoid depends on various factors, such as e.g. the potency of each single compound.

In another aspect the present invention provides a pharmaceutical composition comprising, beside pharmaceutically acceptable excipient, in combination a compound of formula Iₚ and hydrocortisone.

In another aspect the present invention provides a pharmaceutical composition comprising, beside pharmaceutically acceptable excipient, in combination a compound of formula Iₚ and betamethasone, e.g. betamethasone 17-valerate.

In another aspect the present invention provides a pharmaceutical composition comprising, beside pharmaceutically acceptable excipient, in combination a compound of formula Iₚ and dexamethasone.

In a combination according to the present invention, pharmaceutically acceptable excipient, such as appropriate carrier and/or diluent, e.g. includes fillers, binders, disintegrators, flow conditioners, lubricants, sugars and sweeteners, fragrances, preservatives, stabilizers, wetting agents and/or emulsifiers, solubilizers, salts for regulating osmotic pressure, buffers.

A combination of the present invention includes
fixed combinations, in which both pharmaceutically active agents are in the same formulation;
kits, in which both pharmaceutically active agents in separate formulations are sold in the same package, e.g. with instructions for co-administration; and
free combinations in which the pharmaceutically active agents are packaged separately, but instructions for simultaneous or sequential administration are given.

Pharmaceutical compositions of the present invention may be manufactured according, e.g. analogously, to a method as conventional, e.g. by mixing, granulating, coating, dissolving or lyophilizing processes. Unit dosage forms may contain, for example, from about 0.5 mg to about 1000 mg, such as 1 mg to about 500 mg of pharmaceutically active compounds. A pharmaceutical composition of and for use according to the present invention may be administered by any conventional route, for example enterally, e.g. including nasal, buccal, rectal, oral, administration; parenterally, e.g. including intravenous, intramuscular, subcutaneous administration; or topically; e.g. including epicutaneous, intranasal, intratracheal administration;

e.g. in form of coated or uncoated tablets, capsules, injectable solutions or suspensions, e.g. in the form of ampoules, vials, in the form of creams, gels, pastes, inhaler powder, foams, tinctures, lip sticks, drops, sprays, or in the form of suppositories.

Calcineurin inhibitors, including e.g. compounds of formulae I, II and III, and corticoids are known or may be obtained according, e.g. analogously, to a method as conventional.

A combination of the present invention may comprise beside a calcineurin inhibitor and a corticoid as active ingredients further pharmaceutically active compounds. Such further pharmaceutically active compounds include other anti-inflammatory, immunomodulatory and anti-proliferative agents.

In another aspect the present invention provides a pharmaceutical composition of the present invention, further comprising another pharmaceutically active agent.

We have established in vitro systems using human peripheral blood mononuclear cells (PBMC), that serve as in vitro experimental models of resistance to corticoids and/or calcineurin inhibitors, in which a corticoid alone or a calcineurin inhibitor, including e.g. Cyclosporin A and a compound of formula I, II or III, alone exert either no effect at all, or only a partial inhibitory effect on T-cell proliferation. These systems which are described herein as corticoid-resistant and/or calcineurin inhibitor-resistant, e.g. compound of formula I-resistant, employ a high cell density, such as 50,000-200,000 cells/well in a 96-well plate and powerful stimuli of T-cell proliferation, namely the superantigen Staphylococcal Enterotoxin B (SEB) and/or the combination of anti-CD3 plus anti-CD28 monoclonal antibodies. This contrasts with systems employing lower cell densities and/or weaker stimuli, such as e.g. anti-CD3 antibody alone, in which corticoids and calcineurin inhibitors exert a complete, or almost complete, inhibition of T-cell proliferation at nanomolar to sub-nanomolar concentrations.

Surprisingly, in the resistant system described herein we have found that combinations of a calcineurin inhibitor and a corticoid strongly inhibit T-cell proliferation (e.g. 60% inhibition up to complete inhibition), whereas the single components of the combination do not inhibit at all or do not inhibit by more than 35%. Determinations and measurements are as set out under “Methods” in the Examples.

As shown in the Examples, we have found in our in vitro systems that a compound of formula I, II or III, e.g. I_{P}, II_{FK} or Cyclosporin A alone, or hydrocortisone alone, or betamethasone 17-valerate alone, or dexamethasone alone may show either essentially no inhibition or only a partial inhibition, e.g. less than 35% of T-cell proliferation, whereas a combination of hydrocortisone and a compound of formula I_{P}, or a combination of betamethasone 17-valerate and a compound of formula I_{P}, or a combination of dexamethasone and a compound of formula I_{P}, or a combination of hydrocortisone and a compound of formula II_{FK}, or a combination of dexamethasone and a compound of formula II_{FK}, or a combination of hydrocortisone and Cyclosporin A, or a combination of betemethasone 17-valerate and Cyclosporin A, or a combination of dexamethasone and Cyclosporin A in the same assays shows an inhibition of at least 60% up to complete inhibition. We thus have found that a combination of the compounds of the present invention may show activity in cases where the single components do not show inhibition or show inhibition of less than 35%, e.g. less than 25%, such as less than 20%.

In the following examples temperatures are given in degrees Celsius (° C.) and are uncorrected.

The following abbreviations are used:
ASM a compound of formula I_{P} (ASM981, pimecrolimus)
BETA betamethasone 17-valerate
BrdU 5-bromo-2-deoxy-uridine
CsA Cyclosporin A
DEX dexamethasone
DMSO dimethylsulfoxide
FCS fetal calf serum
FK a compound of formula II_{FK} (FK 506, tacrolimus)
HC hydrocortisone
mAb monoclonal antibody
μg microgram
μM micromolar
nM nanomolar
OD Optical Density
PBMC peripheral blood mononuclear cells
rpm revolutions per minute
RPMI medium developed at Roswell Park Memorial Institute
RT room temperature
SD Standard Deviation
SEB Staphylococcal Enterotoxin B (a superantigen)
STIM-C stimulated control
UNSTIM-C unstimulated control

### EXAMPLE A

Materials

The mouse anti-CD3 mAb (clone SPV-T3/1, isotype IgG2a), which stimulates the human T cell receptor, is known (Spits H., Keizer G., Borst J. et al., (1983) Characterization of monoclonal antibodies against cell surface molecules associated with cytotoxic activity of natural and activated killer cells and cloned CTL lines, Hybridoma 2:423-437) and may be prepared as appropriate. The mouse anti-CD28 mAb (clone CD28.2, isotype IgG1, κ) is obtained from BD Biosciences (Catalog #555725). SEB is obtained from Toxin Technology Inc. (Sarasota, Fla., USA; Catalog #TX-BT202). Cell proliferation ELISA kits (colorimetric) based on the measurement of BrdU incorporation during DNA synthesis are obtained from Roche Molecular Biochemicals (Mannheim, Germany). These kits are used according to the manufacturer's instructions.

Cell Cultures

PBMC are isolated from human buffy coats by Ficoll/Hypaque centrifugation under sterile conditions, frozen in 90% FCS+10% DMSO and stored in liquid nitrogen. For each experiment frozen PBMC are thawed and then washed and resuspended in a culture medium consisting of RPMI 1640 (Gibco-BRL, Paisley, UK), supplemented with 10% heat-inactivated FCS (Gibco), 2 mM glutamine, 0.1 mg/ml streptomycin, and 100 units/ml penicillin (Gibco). 0.1 ml of cell suspension obtained are added per well to columns 2-11 of 96-well flat-bottomed cell culture plates (Nunc, Roskilde, Denmark), whereas column 12 contains only medium and serves as the medium blank. Only the inner wells receive cells and are used in the experiments (i.e. column 1 and rows A and H are excluded; receiving only medium). The solvent control and test compounds (0.05 ml/well added) are first incubated with cells at 37° C./5% CO₂ for 2 hours. The stimulant is then added (0.05 ml/well) to all wells to the indicated final concentrations, except to the unstimulated control, which is receiving culture medium (0.05 ml/well), and the plates are incubated for another 68 hours at 37° C./5% CO₂ followed by the addition of BrdU (0.02 ml/well of the BrdU labeling solution [=10 μM BrdU in cell culture medium] from the ELISA kit). After further incubation at 37° C./5% CO₂ for 4 hours, the plates are centrifuged at 1200 rpm (300×g) for 10 min at RT. After removal of the supernatants, the plates are incubated at 60° C. for 1 hour to dry the cells. The solvent control and each compound or compound combination (at the indicated final concentrations) are tested in triplicate (i.e. 3 wells per concentration). Test compounds (i.e. compound-combinations of the present invention and single compounds of such combinations) are dissolved in DMSO and then subsequently diluted into cell culture medium so that the final DMSO concentration in the cell plates does not exceed 0.2% (v/v).

T-Cell Proliferation Assay

The plates are processed for the determination of cell proliferation based on the incorporation of BrdU during DNA synthesis using an ELISA kit (Roche Molecular Biochemicals) according to the manufacturer's instructions. The optical densities are measured in a microtiter plate reader at 450 nm, with a reference wavelength of 690 nm. The absorbance data are analyzed by the software program Excel™. The average of the values in the cell-free wells is used as the blank and subtracted from the other values. The averages and standard deviations of the absorbances for each compound and compound combination are calculated and then normalized to the solvent control containing stimulus, (i.e. stimulated control), which is defined as 100%. The % inhibition for each compound and compound combination is also calculated.

In the following Examples 1 to 27, the T-cell proliferation assay is carried out analogously as described herein.

On day 0 the cells are seeded into 96-well cell culture plates and preincubated with compound(s) for 2 hours before addition of stimulus. On day 3 (i.e. 68 hours later) BrdU is added and the plates are incubated for 4 hours before being processed to determine BrdU incorporation via the BrdU ELISA.

In the TABLES, the following terms are defined:
“Unstimulated Control”: the mean of the OD values measured for the unstimulated control (i.e. cells minus stimulus and compound(s)), along with the calculated SD values.
“Stimulated Control”: the mean of the OD values measured for the stimulated control (i.e. cells plus stimulus and minus compound(s)), along with the calculated SD values.
“Compound(s)”: the mean of the OD values measured for the samples in the presence of stimulus and compound(s), along with the calculated SD values.

“Normalized OD [or SD] (% Stimulated Control)”: the OD and SD values are normalized relative to the Stimulated Control, which is set to 100.

“% Inhibition”: defined as 100%−[(Sample OD−Unstimulated Control OD)/(Stimulated Control OD−Unstimulated Control OD)]×100%. A negative value indicates stimulation of proliferation relative to the stimulated control (defined as 0% inhibition). A value greater than 100 indicates inhibition to below the level of the unstimulated control (defined as 100% inhibition).

### EXAMPLE 1

Cells/Well: 200000

Compound(s): HC (10 μM), ASM (30 nM), BETA (300 nM), a combination of HC (10 μM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 1 below are obtained.

TABLE 1

| | UNSTIM- | STIM- | | | | ASM + | ASM + |
|---|---|---|---|---|---|---|---|
| | C | C | HC | ASM | BETA | HC | BETA |
| OD | 0.068 | 0.768 | 0.820 | 0.721 | 0.808 | 0.300 | 0.228 |
| SD | 0.012 | 0.028 | 0.015 | 0.099 | 0.034 | 0.060 | 0.022 |
| Normalized OD | 8.9 | 100 | 106.8 | 93.9 | 105.2 | 39.1 | 29.7 |
| (% STIM-C) | | | | | | | |
| Normalized SD | 1.6 | 3.6 | 2.0 | 12.9 | 4.4 | 7.8 | 2.9 |
| (% STIM-C) | | | | | | | |
| % Inhibition | 100 | 0 | −7.4 | 6.7 | −5.7 | 66.9 | 77.1 |

From TABLE 1 it is evident that ASM alone, HC alone and BETA alone show essentially no inhibition of T-cell proliferation in this system, whereas combinations of ASM+HC and ASM+BETA show inhibition of more than 65%.

### EXAMPLE 2

Cells/Well: 100000

Compound(s): HC (10 μM), ASM (30 nM), BETA (300 nM), a combination of HC (10 μM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 2 below are obtained.

TABLE 2

| | UNSTIM-C | STIM-C | HC | ASM | BETA | ASM + HC | ASM + BETA |
|---|---|---|---|---|---|---|---|
| OD | 0.082 | 0.912 | 0.839 | 0.776 | 0.796 | 0.301 | 0.189 |
| SD | 0.003 | 0.085 | 0.055 | 0.091 | 0.126 | 0.015 | 0.046 |
| Normalized OD | 9.0 | 100 | 92.0 | 85.1 | 87.3 | 33.0 | 20.7 |
| (% STIM-C) | | | | | | | |
| Normalized SD | 0.3 | 9.3 | 6.0 | 10.0 | 13.8 | 1.6 | 5.0 |
| (% STIM-C) | | | | | | | |
| % Inhibition | 100 | 0 | 8.8 | 16.4 | 14.0 | 73.6 | 87.1 |

From TABLE 2 it is evident that ASM alone, HC alone and BETA alone show less than 17% inhibition of T-cell proliferation in this system, whereas combinations of ASM+HC and ASM+BETA show inhibitions of more than 73%.

### EXAMPLE 3

Cells/Well: 50000

Compound(s): HC (10 μM), ASM (30 nM), BETA (300 nM), a combination of HC (10 μM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 3 below are obtained.

TABLE 3

| | UNSTIM-C | STIM-C | HC | ASM | BETA | ASM + HC | ASM + BETA |
|---|---|---|---|---|---|---|---|
| OD | 0.065 | 0.878 | 0.957 | 0.994 | 0.873 | 0.115 | 0.102 |
| SD | 0.012 | 0.087 | 0.053 | 0.039 | 0.075 | 0.017 | 0.035 |
| Normalized OD | 7.4 | 100 | 109.0 | 113.2 | 99.4 | 13.1 | 11.6 |
| (% STIM-C) | | | | | | | |
| Normalized SD | 1.4 | 9.9 | 6.0 | 4.4 | 8.5 | 1.9 | 4.0 |
| (% STIM-C) | | | | | | | |
| % Inhibition | 100 | 0 | −9.7 | −14.3 | 0.6 | 93.8 | 95.4 |

From TABLE 3 it is evident that ASM alone, HC alone and BETA alone show essentially no inhibition of T-cell proliferation in this system, whereas combinations of ASM+HC and ASM+BETA show inhibitions of more than 93%.

### EXAMPLE 4

Cells/Well: 200000

Compound(s): HC (10 μM), ASM (30 nM), BETA (300 nM), a combination of HC (10 μM)+ASM (30 nM) and a combination of BETA (300 nM)+ASM (30 nM)

Stimulus: SEB (0.1 μg/ml)

Results: as set out in TABLE 4 below are obtained.

TABLE 4

| | UNSTIM-C | STIM-C | HC | ASM | BETA | ASM + HC | ASM + BETA |
|---|---|---|---|---|---|---|---|
| OD | 0.069 | 1.059 | 0.942 | 1.015 | 0.900 | 0.070 | 0.073 |
| SD | 0.006 | 0.204 | 0.078 | 0.065 | 0.095 | 0.012 | 0.010 |
| Normalized OD | 6.5 | 100 | 89.0 | 95.8 | 85.0 | 6.6 | 6.9 |
| (% STIM-C) | | | | | | | |
| Normalized SD | 0.6 | 19.3 | 7.4 | 6.1 | 9.0 | 1.1 | 0.9 |
| (% STIM-C) | | | | | | | |
| % Inhibition | 100 | 0 | 11.8 | 4.4 | 16.1 | 99.9 | 99.6 |

From TABLE 4 it is evident that ASM alone, HC alone and BETA alone show less than 17% inhibition of T-cell proliferation in this system, whereas combinations of ASM+HC and ASM+BETA show inhibitions of more than 99% (i.e. essentially complete inhibition).

### EXAMPLE 5

Cells/Well: 100000

Compound(s): HC (10 μM), ASM (30 nM), BETA (300 nM), a combination of HC (10 μM)+ASM (30 nM) and a combination of BETA (300 nM)+ASM (30 nM)

Stimulus: SEB (0.1 μg/ml)

Results: as set out in TABLE 5 below are obtained.

TABLE 5

| | UNSTIM-C | STIM-C | HC | ASM | BETA | ASM + HC | ASM + BETA |
|---|---|---|---|---|---|---|---|
| OD | 0.050 | 0.851 | 0.855 | 0.665 | 1.001 | 0.051 | 0.043 |
| SD | 0.022 | 0.057 | 0.100 | 0.196 | 0.145 | 0.017 | 0.001 |
| Normalized OD | 5.9 | 100 | 100.5 | 78.1 | 117.6 | 6.0 | 5.1 |
| (% STIM-C) | | | | | | | |
| Normalized SD | 2.6 | 6.7 | 11.8 | 23.0 | 17.0 | 2.0 | 0.1 |
| (% STIM-C) | | | | | | | |
| % Inhibition | 100 | 0 | −0.5 | 23.2 | −18.7 | 99.9 | 100.9 |

From TABLE 5 it is evident that ASM alone, HC alone and BETA alone show less than 24% inhibition of T-cell proliferation in this system, whereas combinations of ASM+HC and ASM+BETA show inhibitions of more than 99% (i.e. essentially complete inhibition).

### EXAMPLE 6

Cells/Well: 200000

Compound(s): DEX (300 nM), ASM (30 nM), BETA (300 nM), a combination of DEX (300 nM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 6 below are obtained.

TABLE 6

| | UNSTIM-C | STIM-C | DEX | ASM | BETA | ASM + DEX | ASM + BETA |
|---|---|---|---|---|---|---|---|
| OD | 0.022 | 0.788 | 0.687 | 0.772 | 0.703 | 0.145 | 0.179 |
| SD | 0.003 | 0.048 | 0.026 | 0.152 | 0.027 | 0.026 | 0.035 |
| Normalized OD | 2.8 | 100 | 87.2 | 98.0 | 89.2 | 18.4 | 22.7 |
| (% STIM-C) | | | | | | | |
| Normalized SD | 0.4 | 6.1 | 3.3 | 19.3 | 3.4 | 3.3 | 4.4 |
| (% STIM-C) | | | | | | | |
| % Inhibition | 100 | 0 | 13.2 | 2.1 | 11.1 | 83.9 | 79.5 |

From TABLE 6 it is evident that ASM alone, DEX alone and BETA alone show less than 14% inhibition of T-cell proliferation in this system, whereas combinations of ASM+DEX and ASM+BETA show inhibitons of more than 79%.

### EXAMPLE 7

Cells/Well: 100000

Compound(s): DEX (300 nM), ASM (30 nM), BETA (300 nM), a combination of DEX (300 nM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 7 below are obtained.

TABLE 7

| | UNSTIM-C | STIM-C | DEX | ASM | BETA | ASM + DEX | ASM + BETA |
|---|---|---|---|---|---|---|---|
| OD | 0.024 | 0.840 | 0.643 | 0.734 | 0.692 | 0.134 | 0.084 |
| SD | 0.009 | 0.052 | 0.020 | 0.051 | 0.118 | 0.071 | 0.011 |
| Normalized OD | 2.9 | 100 | 76.5 | 87.4 | 82.4 | 16.0 | 10.0 |
| (% STIM-C) | | | | | | | |
| Normalized SD | 1.1 | 6.2 | 2.4 | 6.1 | 14.0 | 8.5 | 1.3 |
| (% STIM-C) | | | | | | | |
| % Inhibition | 100 | 0 | 24.1 | 13.0 | 18.1 | 86.5 | 92.6 |

From TABLE 7 it is evident that ASM alone, DEX alone and BETA alone show less than 25% inhibition of T-cell proliferation in this system, whereas combinations of ASM+DEX and ASM+BETA show inhibitions of more than 86%.

### EXAMPLE 8

Cells/Well: 50000

Compound(s): DEX (300 nM), ASM (30 nM), BETA (300 nM), a combination of DEX (300 nM)+ASM (30 nM) and a combination of BETA (300 nM)+ASM (30 nM)

Stimulus: anti-CD3 mAb (0.1 μg/ml)+anti-CD28 mAb (1 μg/ml)

Results: as set out in TABLE 8 below are obtained.

TABLE 8

| | UNSTIM-C | STIM-C | DEX | ASM | BETA | ASM + DEX | ASM + BETA |
|---|---|---|---|---|---|---|---|
| OD | 0.023 | 1.053 | 0.866 | 0.908 | 0.794 | 0.056 | 0.057 |
| SD | 0.004 | 0.133 | 0.083 | 0.146 | 0.136 | 0.018 | 0.003 |
| Normalized OD | 2.2 | 100 | 82.2 | 86.2 | 75.4 | 5.3 | 5.4 |
| (% STIM-C) | | | | | | | |
| Normalized SD | 0.4 | 12.6 | 7.9 | 13.9 | 12.9 | 1.7 | 0.3 |
| (% STIM-C) | | | | | | | |
| % Inhibition | 100 | 0 | 18.2 | 14.1 | 25.1 | 96.8 | 96.7 |

From TABLE 8 it is evident that ASM alone, DEX alone and BETA alone show less than 26% inhibition of T-cell proliferation in this system, whereas combinations of ASM+DEX and ASM+BETA show inhibitions of more than 96%.

### EXAMPLE 9

Cells/Well: 200000

Compound(s): DEX (300 nM), ASM (30 nM), BETA (300 nM), a combination of DEX (300 nM)+ASM (30 nM) and a combination of BETA (300 nM)+ASM (30 nM)

Stimulus: SEB (0.1 μg/ml)

Results: as set out in TABLE 9 below are obtained.

TABLE 9

| | UNSTIM-C | STIM-C | DEX | ASM | BETA | ASM + DEX | ASM + BETA |
|---|---|---|---|---|---|---|---|
| OD | 0.065 | 0.905 | 0.919 | 0.752 | 0.902 | 0.052 | 0.059 |
| SD | 0.012 | 0.070 | 0.008 | 0.057 | 0.130 | 0.010 | 0.004 |
| Normalized OD | 7.2 | 100 | 101.5 | 83.1 | 99.7 | 5.7 | 6.5 |
| (% STIM-C) | | | | | | | |
| Normalized SD | 1.3 | 7.7 | 0.9 | 6.3 | 14.4 | 1.1 | 0.4 |
| (% STIM-C) | | | | | | | |
| % Inhibition | 100 | 0 | −1.7 | 18.2 | 0.4 | 101.5 | 100.7 |

From TABLE 9 it is evident that ASM alone, DEX alone and BETA alone show less than 19% inhibition of T-cell proliferation in this system, whereas combinations of ASM+DEX and ASM+BETA show inhibitions of more than 100% (i.e. essentially complete inhibition).

### EXAMPLE 10

Cells/Well: 100000

Compound(s): DEX (300 nM), ASM (30 nM), BETA (300 nM), a combination of DEX (300 nM)+ASM (30 nM) and a combination of BETA (300 nM)+ASM (30 nM)

Stimulus: SEB (0.1 μg/ml)

Results: as set out in TABLE 10 below are obtained.

TABLE 10

| | UNSTIM-C | STIM-C | DEX | ASM | BETA | ASM + DEX | ASM + BETA |
|---|---|---|---|---|---|---|---|
| OD | 0.022 | 0.980 | 0.847 | 0.660 | 0.955 | 0.023 | 0.029 |
| SD | 0.008 | 0.153 | 0.084 | 0.138 | 0.053 | 0.008 | 0.005 |
| Normalized OD | 2.2 | 100 | 86.4 | 67.3 | 97.4 | 2.3 | 3.0 |
| (% STIM-C) | | | | | | | |
| Normalized SD | 0.8 | 15.6 | 8.6 | 14.1 | 5.4 | 0.8 | 0.5 |
| (% STIM-C) | | | | | | | |
| % Inhibition | 100 | 0 | 13.9 | 33.4 | 2.6 | 99.9 | 99.3 |

From TABLE 10 it is evident that ASM alone, DEX alone and BETA alone show less than 34% inhibition of T-cell proliferation in this system, whereas combinations of ASM+DEX and ASM+BETA show inhibitions of more than 99% (i.e. essentially complete inhibition).

### EXAMPLE 11

Cells/Well: 100000

Compound(s): DEX (300 nM), ASM (30 nM), and a combination of DEX (300 nM)+ASM (30 nM)

Stimulus: anti-CD3 mAb (0.1 μg/ml)+anti-CD28 mAb (1 μg/ml)

Results: as set out in TABLE 11 below are obtained.

TABLE 11

| | | | | | ASM + |
|---|---|---|---|---|---|
| | UNSTIM-C | STIM-C | DEX | ASM | DEX |
| OD | 0.033 | 1.160 | 0.885 | 0.918 | 0.069 |
| SD | 0.008 | 0.060 | 0.048 | 0.031 | 0.007 |
| Normalized OD | 2.8 | 100 | 76.3 | 79.1 | 5.9 |
| (% STIM-C) | | | | | |
| Normalized SD | 0.7 | 5.2 | 4.1 | 2.7 | 0.6 |
| (% STIM-C) | | | | | |
| % Inhibition | 100 | 0 | 24.4 | 21.5 | 96.8 |

From TABLE 11 it is evident that ASM alone and DEX alone show less than 25% inhibition of T-cell proliferation in this system, whereas a combination of ASM+DEX shows an inhibition of more than 96%.

### EXAMPLE 12

Cells/Well: 100000

Compound(s): DEX (300 nM), ASM (30 nM), and a combination of DEX (300 nM)+ASM (30 nM)

Stimulus: SEB (0.1 μg/ml)

Results: as set out in TABLE 12 below are obtained.

TABLE 12

| | | | | | ASM + |
|---|---|---|---|---|---|
| | UNSTIM-C | STIM-C | DEX | ASM | DEX |
| OD | 0.044 | 1.307 | 1.382 | 1.013 | 0.022 |
| SD | 0.003 | 0.183 | 0.050 | 0.135 | 0.003 |
| Normalized OD | 3.4 | 100 | 105.7 | 77.5 | 1.7 |
| (% STIM-C) | | | | | |
| Normalized SD | 0.2 | 14.0 | 3.8 | 10.3 | 0.2 |
| (% STIM-C) | | | | | |
| % Inhibition | 100 | 0 | −5.9 | 23.3 | 101.7 |

From TABLE 12 it is evident that ASM alone and DEX alone show less than 24% inhibition of T-cell proliferation in this system, whereas a combination of ASM+DEX shows an inhibition of more than 101% (i.e. essentially complete inhibition).

### EXAMPLE 13

Cells/Well: 200000

Compound(s): HC (10 μM), FK (30 nM), BETA (300 nM), a combination of HC (10 μM)+FK (30 nM) and a combination of BETA (300 nM)+FK (30 nM)

Stimulus: anti-CD3 mAb (0.1 μg/ml)+anti-CD28 mAb (1 μg/ml)

Results: as set out in TABLE 13 below are obtained.

TABLE 13

| | | | | | | FK + | FK + |
|---|---|---|---|---|---|---|---|
| | UNSTIM-C | STIM-C | HC | FK | BETA | HC | BETA |
| OD | 0.068 | 0.768 | 0.820 | 0.921 | 0.808 | 0.345 | 0.152 |
| SD | 0.012 | 0.028 | 0.015 | 0.063 | 0.034 | 0.155 | 0.054 |
| Normalized OD | 8.9 | 100 | 106.8 | 119.9 | 105.2 | 44.9 | 19.8 |
| (% STIM-C) | | | | | | | |
| Normalized SD | 1.6 | 3.6 | 2.0 | 8.2 | 4.4 | 20.2 | 7.0 |
| (% STIM-C) | | | | | | | |
| % Inhibition | 100 | 0 | −7.4 | −21.9 | −5.7 | 60.4 | 88.0 |

From TABLE 13 it is evident that FK alone, HC alone and BETA alone show essentially no inhibition of T-cell proliferation in this system, whereas combinations of FK+HC and FK+BETA show inhibition of more than 60%.

### EXAMPLE 14

Cells/Well: 100000

Compound(s): HC (10 μM), FK (30 nM), BETA (300 nM), a combination of HC (10 μM)+FK (30 nM) and a combination of BETA (300 nM)+FK (30 nM)

Stimulus: anti-CD3 mAb (0.1 μg/ml)+anti-CD28 mAb (1 μg/ml) Results: as set out in TABLE 14 below are obtained.

TABLE 14

| | UNSTIM-C | STIM-C | HC | FK | BETA | FK + HC | FK + BETA |
|---|---|---|---|---|---|---|---|
| OD | 0.082 | 0.912 | 0.839 | 0.662 | 0.796 | 0.243 | 0.101 |
| SD | 0.003 | 0.085 | 0.055 | 0.147 | 0.126 | 0.046 | 0.032 |
| Normalized OD | 9.0 | 100 | 92.0 | 72.6 | 87.3 | 26.6 | 11.1 |
| (% STIM-C) | | | | | | | |
| Normalized SD | 0.3 | 9.3 | 6.0 | 16.1 | 13.8 | 5.0 | 3.5 |
| (% STIM-C) | | | | | | | |
| % Inhibition | 100 | 0 | 8.8 | 30.1 | 14.0 | 80.6 | 97.7 |

From TABLE 14 it is evident that FK alone, HC alone and BETA alone show less than 31% inhibition of T-cell proliferation in this system, whereas combinations of FK+HC and FK+BETA show inhibitions of more than 80%.

### EXAMPLE 15

Cells/Well: 200000

Compound(s): DEX (300 nM), FK (30 nM), BETA (300 nM), a combination of DEX (300 nM)+FK (30 nM) and a combination of BETA (300 nM)+FK (30 nM)

Stimulus: anti-CD3 mAb (0.1 μg/ml)+anti-CD28 mAb (1 μg/ml)

Results: as set out in TABLE 15 below are obtained.

TABLE 15

| | UNSTIM-C | STIM-C | DEX | FK | BETA | FK + DEX | FK + BETA |
|---|---|---|---|---|---|---|---|
| OD | 0.022 | 0.788 | 0.687 | 0.795 | 0.703 | 0.081 | 0.107 |
| SD | 0.003 | 0.048 | 0.026 | 0.028 | 0.027 | 0.007 | 0.022 |
| Normalized OD | 2.8 | 100 | 87.2 | 100.9 | 89.2 | 10.3 | 13.6 |
| (% STIM-C) | | | | | | | |
| Normalized SD | 0.4 | 6.1 | 3.3 | 3.6 | 3.4 | 0.9 | 2.8 |
| (% STIM-C) | | | | | | | |
| % Inhibition | 100 | 0 | 13.2 | −0.9 | 11.1 | 92.3 | 88.9 |

From TABLE 15 it is evident that FK alone, DEX alone and BETA alone show less than 14% inhibition of T-cell proliferation in this system, whereas combinations of FK+DEX and FK+BETA show inhibitions of more than 88%.

### EXAMPLE 16

Cells/Well: 100000

Compound(s): DEX (300 nM), FK (30 nM), BETA (300 nM), a combination of DEX (300 nM)+FK (30 nM) and a combination of BETA (300 nM)+FK (30 nM)

Stimulus: anti-CD3 mAb (0.1 μg/ml)+anti-CD28 mAb (1 μg/ml)

Results: as set out in TABLE 16 below are obtained.

TABLE 16

| | UNSTIM-C | STIM-C | DEX | FK | BETA | FK + DEX | FK + BETA |
|---|---|---|---|---|---|---|---|
| OD | 0.024 | 0.840 | 0.643 | 0.809 | 0.692 | 0.072 | 0.086 |
| SD | 0.009 | 0.052 | 0.020 | 0.034 | 0.118 | 0.024 | 0.011 |
| Normalized OD | 2.9 | 100 | 76.5 | 96.3 | 82.4 | 8.6 | 10.2 |
| (% STIM-C) | | | | | | | |
| Normalized SD | 1.1 | 6.2 | 2.4 | 4.0 | 14.0 | 2.9 | 1.3 |
| (% STIM-C) | | | | | | | |
| % Inhibition | 100 | 0 | 24.1 | 3.8 | 18.1 | 94.1 | 92.4 |

From TABLE 16 it is evident that FK alone, DEX alone and BETA alone show less than 25% inhibition of T-cell proliferation in this system, whereas combinations of FK+DEX and FK+BETA show inhibitions of more than 92%.

### EXAMPLE 17

Cells/Well: 50000

Compound(s): DEX (300 nM), FK (30 nM), BETA (300 nM), a combination of DEX (300 nM)+FK (30 nM) and a combination of BETA (300 nM)+FK (30 nM)

Stimulus: anti-CD3 mAb (0.1 μg/ml)+anti-CD28 mAb (1 μg/ml)

Results: as set out in TABLE 17 below are obtained.

TABLE 17

| | UNSTIM-C | STIM-C | DEX | FK | BETA | FK + DEX | FK + BETA |
|---|---|---|---|---|---|---|---|
| OD | 0.023 | 1.053 | 0.866 | 0.838 | 0.794 | 0.030 | 0.032 |
| SD | 0.004 | 0.133 | 0.083 | 0.057 | 0.136 | 0.003 | 0.013 |
| Normalized OD | 2.2 | 100 | 82.2 | 79.6 | 75.4 | 2.8 | 3.0 |
| (% STIM-C) | | | | | | | |
| Normalized SD | 0.4 | 12.6 | 7.9 | 5.4 | 12.9 | 0.3 | 1.2 |
| (% STIM-C) | | | | | | | |
| % Inhibition | 100 | 0 | 18.2 | 20.9 | 25.1 | 99.3 | 99.1 |

From TABLE 17 it is evident that FK alone, DEX alone and BETA alone show less than 26% inhibition of T-cell proliferation in this system, whereas combinations of FK+DEX and FK+BETA show inhibitions of more than 99% (i.e. essentially complete inhibition).

### EXAMPLE 18

Cells/Well: 100000

Compound(s): DEX (300 nM), FK (30 nM), and a combination of DEX (300 nM)+FK (30 nM)

Stimulus: anti-CD3 mAb (0.1 μg/ml)+anti-CD28 mAb (1 μg/ml) Results: as set out in TABLE 18 below are obtained.

TABLE 18

| | | | | | FK + |
|---|---|---|---|---|---|
| | UNSTIM-C | STIM-C | DEX | FK | DEX |
| OD | 0.033 | 1.160 | 0.885 | 0.905 | 0.048 |
| SD | 0.008 | 0.060 | 0.048 | 0.101 | 0.006 |
| Normalized OD | 2.8 | 100 | 76.3 | 78.0 | 4.1 |
| (% STIM-C) | | | | | |
| Normalized SD | 0.7 | 5.2 | 4.1 | 8.7 | 0.5 |
| (% STIM-C) | | | | | |
| % Inhibition | 100 | 0 | 24.4 | 22.6 | 98.7 |

From TABLE 18 it is evident that FK alone and DEX alone show less than 25% inhibition of T-cell proliferation in this system, whereas a combination of FK+DEX shows an inhibition of more than 98% (i.e. essentially complete inhibition).

### EXAMPLE 19

Cells/Well: 200000

Compound(s): HC (10 μM), CsA (300 nM), BETA (300 nM), a combination of HC (10 μM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 19 below are obtained.

TABLE 19

| | UNSTIM-C | STIM-C | HC | CsA | BETA | CsA + HC | CsA + BETA |
|---|---|---|---|---|---|---|---|
| OD | 0.068 | 0.768 | 0.820 | 0.760 | 0.808 | 0.239 | 0.154 |
| SD | 0.012 | 0.028 | 0.015 | 0.048 | 0.034 | 0.025 | 0.045 |
| Normalized OD | 8.9 | 100 | 106.8 | 99.0 | 105.2 | 31.1 | 20.1 |
| (% STIM-C) | | | | | | | |
| Normalized SD | 1.6 | 3.6 | 2.0 | 6.3 | 4.4 | 3.3 | 5.9 |
| (% STIM-C) | | | | | | | |
| % Inhibition | 100 | 0 | −7.4 | 1.1 | −5.7 | 75.6 | 87.7 |

From TABLE 19 it is evident that CsA alone, HC alone and BETA alone show essentially no inhibition of T-cell proliferation in this system, whereas combinations of CsA+HC and CsA+BETA show inhibition of more than 75%.

### EXAMPLE 20

Cells/Well: 100000

Compound(s): HC (10 μM), CsA (300 nM), BETA (300 nM), a combination of HC (10 μM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 20 below are obtained.

TABLE 20

| | UNSTIM-C | STIM-C | HC | CsA | BETA | CsA + HC | CsA + BETA |
|---|---|---|---|---|---|---|---|
| OD | 0.082 | 0.912 | 0.839 | 0.783 | 0.796 | 0.167 | 0.091 |
| SD | 0.003 | 0.085 | 0.055 | 0.119 | 0.126 | 0.039 | 0.015 |
| Normalized OD | 9.0 | 100 | 92.0 | 85.9 | 87.3 | 18.3 | 10.0 |
| (% STIM-C) | | | | | | | |
| Normalized SD | 0.3 | 9.3 | 6.0 | 13.0 | 13.8 | 4.3 | 1.6 |
| (% STIM-C) | | | | | | | |
| % Inhibition | 100 | 0 | 8.8 | 15.5 | 14.0 | 89.8 | 98.9 |

From TABLE 20 it is evident that CsA alone, HC alone and BETA alone show less than 16% inhibition of T-cell proliferation in this system, whereas combinations of CsA+HC and CsA+BETA show inhibitions of more than 89%.

### EXAMPLE 21

Cells/Well: 50000

Compound(s): HC (10 μM), CsA (300 nM), BETA (300 nM), a combination of HC (10 μM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 21 below are obtained.

TABLE 21

| | UNSTIM-C | STIM-C | HC | CsA | BETA | CsA + HC | CsA + BETA |
|---|---|---|---|---|---|---|---|
| OD | 0.065 | 0.878 | 0.957 | 0.865 | 0.873 | 0.082 | 0.055 |
| SD | 0.012 | 0.087 | 0.053 | 0.039 | 0.075 | 0.024 | 0.014 |
| Normalized OD | 7.4 | 100 | 109.0 | 98.5 | 99.4 | 9.3 | 6.3 |
| (% STIM-C) | | | | | | | |
| Normalized SD | 1.4 | 9.9 | 6.0 | 4.4 | 8.5 | 2.7 | 1.6 |
| (% STIM-C) | | | | | | | |
| % Inhibition | 100 | 0 | −9.7 | 1.6 | 0.6 | 97.9 | 101.2 |

From TABLE 21 it is evident that CsA alone, HC alone and BETA alone show essentially no inhibition of T-cell proliferation in this system, whereas combinations of CsA+HC and CsA+BETA show inhibitions of more than 97% (i.e. essentially complete inhibition).

### EXAMPLE 22

Cells/Well: 200000

Compound(s): HC (10 μM), CsA (300 nM), BETA (300 nM), a combination of HC (10 μM)+CsA (30 nM) and a combination of BETA (300 nM)+CsA (300 nM)

Stimulus: SEB (0.1 μg/ml)

Results: as set out in TABLE 22 below are obtained.

TABLE 22

| | UNSTIM-C | STIM-C | HC | CsA | BETA | CsA + HC | CsA + BETA |
|---|---|---|---|---|---|---|---|
| OD | 0.069 | 1.059 | 0.942 | 1.122 | 0.900 | 0.061 | 0.074 |
| SD | 0.006 | 0.204 | 0.078 | 0.241 | 0.095 | 0.005 | 0.008 |
| Normalized OD | 6.5 | 100 | 89.0 | 105.9 | 85.0 | 5.8 | 7.0 |
| (% STIM-C) | | | | | | | |
| Normalized SD | 0.6 | 19.3 | 7.4 | 22.8 | 9.0 | 0.5 | 0.8 |
| (% STIM-C) | | | | | | | |
| % Inhibition | 100 | 0 | 11.8 | −6.4 | 16.1 | 100.8 | 99.5 |

From TABLE 22 it is evident that CsA alone, HC alone and BETA alone show less than 17% inhibition of T-cell proliferation in this system, whereas combinations of CsA+HC and CsA+BETA show inhibitions of more than 99% (i.e. essentially complete inhibition).

### EXAMPLE 23

Cells/Well: 200000

Compound(s): DEX (300 nM), CsA (300 nM), BETA (300 nM), a combination of DEX (300 nM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 23 below are obtained.

TABLE 23

| | UNSTIM-C | STIM-C | DEX | CsA | BETA | CsA + DEX | CsA + BETA |
|---|---|---|---|---|---|---|---|
| OD | 0.022 | 0.788 | 0.687 | 0.786 | 0.703 | 0.110 | 0.102 |
| SD | 0.003 | 0.048 | 0.026 | 0.088 | 0.027 | 0.027 | 0.028 |
| Normalized OD | 2.8 | 100 | 87.2 | 99.7 | 89.2 | 14.0 | 12.9 |
| (% STIM-C) | | | | | | | |
| Normalized SD | 0.4 | 6.1 | 3.3 | 11.2 | 3.4 | 3.4 | 3.6 |
| (% STIM-C) | | | | | | | |
| % Inhibition | 100 | 0 | 13.2 | 0.3 | 11.1 | 88.5 | 89.6 |

From TABLE 23 it is evident that CsA alone, DEX alone and BETA alone show less than 14% inhibition of T-cell proliferation in this system, whereas combinations of CsA+DEX and CsA+BETA show inhibitions of more than 88%.

### EXAMPLE 24

Cells/Well: 100000

Compound(s): DEX (300 nM), CsA (300 nM), BETA (300 nM), a combination of DEX (300 nM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 24 below are obtained.

TABLE 24

| | UNSTIM-C | STIM-C | DEX | CsA | BETA | CsA + DEX | CsA + BETA |
|---|---|---|---|---|---|---|---|
| OD | 0.024 | 0.840 | 0.643 | 0.804 | 0.692 | 0.070 | 0.061 |
| SD | 0.009 | 0.052 | 0.020 | 0.022 | 0.118 | 0.017 | 0.006 |
| Normalized OD | 2.9 | 100 | 76.5 | 95.7 | 82.4 | 8.3 | 7.3 |
| (% STIM-C) | | | | | | | |
| Normalized SD | 1.1 | 6.2 | 2.4 | 2.6 | 14.0 | 2.0 | 0.7 |
| (% STIM-C) | | | | | | | |
| % Inhibition | 100 | 0 | 24.1 | 4.4 | 18.1 | 94.4 | 95.5 |

From TABLE 24 it is evident that CsA alone, DEX alone and BETA alone show less than 25% inhibition of T-cell proliferation in this system, whereas combinations of CsA+DEX and CsA+BETA show inhibitions of more than 94%.

### EXAMPLE 25

Cells/Well: 50000

Compound(s): DEX (300 nM), CsA (300 nM), BETA (300 nM), a combination of DEX (300 nM)+CsA (30 nM) and a combination of BETA (300 nM)+CsA (300 nM)

Stimulus: anti-CD3 mAb (0.1 μg/ml)+anti-CD28 mAb (1 μg/ml)

Results: as set out in TABLE 25 below are obtained.

TABLE 25

| | UNSTIM-C | STIM-C | DEX | CsA | BETA | CsA + DEX | CsA + BETA |
|---|---|---|---|---|---|---|---|
| OD | 0.023 | 1.053 | 0.866 | 0.869 | 0.794 | 0.033 | 0.039 |
| SD | 0.004 | 0.133 | 0.083 | 0.026 | 0.136 | 0.010 | 0.013 |
| Normalized OD | 2.2 | 100 | 82.2 | 82.5 | 75.4 | 3.1 | 3.7 |
| (% STIM-C) | | | | | | | |
| Normalized SD | 0.4 | 12.6 | 7.9 | 2.5 | 12.9 | 0.9 | 1.2 |
| (% STIM-C) | | | | | | | |
| % Inhibition | 100 | 0 | 18.2 | 17.9 | 25.1 | 99.0 | 98.4 |

From TABLE 25 it is evident that CsA alone, DEX alone and BETA alone show less than 26% inhibition of T-cell proliferation in this system, whereas combinations of CsA+DEX and CsA+BETA show inhibitions of more than 98% (i.e. essentially complete inhibition).

### EXAMPLE 26

Cells/Well: 200000

Compound(s): DEX (300 nM), CsA (300 nM), BETA (300 nM), a combination of DEX (300 nM)+CsA (30 nM) and a combination of BETA (300 nM)+CsA (300 nM)

Stimulus: SEB (0.1 μg/ml)

Results: as set out in TABLE 26 below are obtained.

TABLE 26

| | UNSTIM-C | STIM-C | DEX | CsA | BETA | CsA + DEX | CsA + BETA |
|---|---|---|---|---|---|---|---|
| OD | 0.065 | 0.905 | 0.919 | 0.777 | 0.902 | 0.052 | 0.064 |
| SD | 0.012 | 0.070 | 0.008 | 0.083 | 0.130 | 0.025 | 0.016 |
| Normalized OD | 7.2 | 100 | 101.5 | 85.9 | 99.7 | 5.7 | 7.1 |
| (% STIM-C) | | | | | | | |
| Normalized SD | 1.3 | 7.7 | 0.9 | 9.2 | 14.4 | 2.8 | 1.8 |
| (% STIM-C) | | | | | | | |
| % Inhibition | 100 | 0 | −1.7 | 15.2 | 0.4 | 101.5 | 100.1 |

From TABLE 26 it is evident that CsA alone, DEX alone and BETA alone show less than 16% inhibition of T-cell proliferation in this system, whereas combinations of CsA+DEX and CsA+BETA show inhibitions of more than 100% (i.e. essentially complete inhibition).

### EXAMPLE 27

Cells/Well: 100000

Compound(s): DEX (300 nM), CsA (300 nM), and a combination of DEX (300 nM)+CsA (300 nM)

Stimulus: anti-CD3 mAb (0.1 μg/ml)+anti-CD28 mAb (1 μg/ml)

Results: as set out in TABLE 27 below are obtained.

TABLE 27

| | UNSTIM-C | STIM-C | DEX | CsA | CsA + DEX |
|---|---|---|---|---|---|
| OD | 0.033 | 1.160 | 0.885 | 0.844 | 0.048 |
| SD | 0.008 | 0.060 | 0.048 | 0.014 | 0.010 |
| Normalized | 2.8 | 100 | 76.3 | 72.8 | 4.1 |
| OD | | | | | |
| (% STIM-C) | | | | | |
| Normalized SD | 0.7 | 5.2 | 4.1 | 1.2 | 0.9 |
| (% STIM-C) | | | | | |
| % Inhibition | 100 | 0 | 24.4 | 28.0 | 98.7 |

From TABLE 27 it is evident that CsA alone and DEX alone show less than 29% inhibition of T-cell proliferation in this system, whereas a combination of CsA+DEX shows an inhibition of more than 98% (i.e. essentially complete inhibition).

1. Use of a combination of a corticoid and a compound of formula 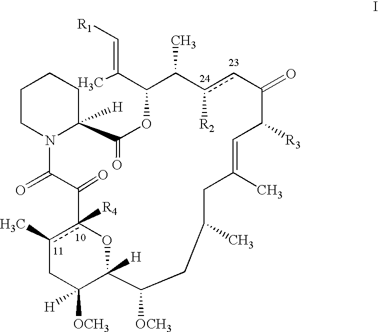
wherein either
R₁ is a group (a) of formula 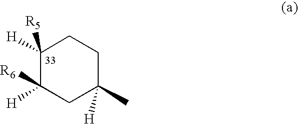
wherein
R₅ is chloro, bromo, iodo or azido,
R₆ is hydroxy or methoxy, and
R₄ is hydroxy and there is a single bond in 10,11 position; or absent, and there is a double bond in 10,11 position or
R₁ is a group selected from 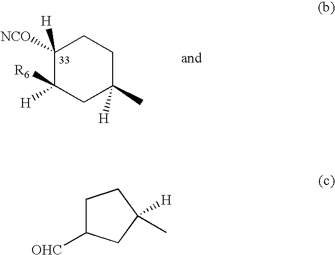
wherein
R₆ is as defined above, and
R₄ is hydroxy and there is a single bond in 10,11 position,
R₂ is oxo and there is a single bond in 23,24 position; hydroxy and there is a single or double bond in 23,24 position; or absent and there is a double bond in 23,24 position;
R₃ is methyl, ethyl, propyl or allyl, for the manufacture of a medicament for the treatment of a corticoid-resistant disease and/or a calcineurin inhibitor-resistant disease.

2. Use of claim 1, wherein the compound of formula I is a compound of formula

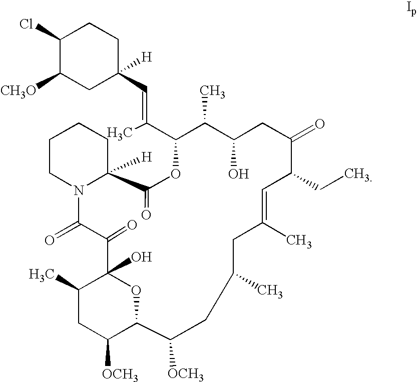

3. Use of claim 1, wherein the corticoid is selected from the group consisting of hydrocortisone, betamethasone and dexamethasone.

4. Use of claim 1, wherein T cells are involved in the pathophysiology of the disease.

5. Use of claim 1, wherein the disease is selected from the group consisting of atopic dermatitis, psoriasis, psoriatic arthritis, rheumatoid arthritis, asthma, ulcerative colitis and Crohn's disease.

6. Use of a combination of a corticoid and a calcineurin inhibitor for the manufacture of a medicament for the treatment of a corticoid-resistant disease and/or a calcineurin inhibitor-resistant disease wherein T cells are involved in the pathophysiology of the disease, with the proviso that focal segmental glomerulosclerosis wherein T cells are involved in the pathophysiology are excluded.

7. Use of claim 6, wherein the calcineurin inhibitor is a compound of formula 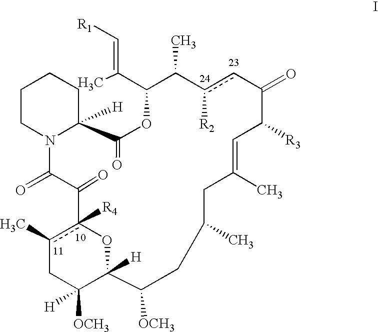
wherein substituents are as defined in claim 1.

8. Use of claim 6, wherein the calcineurin inhibitor is a compound of formula 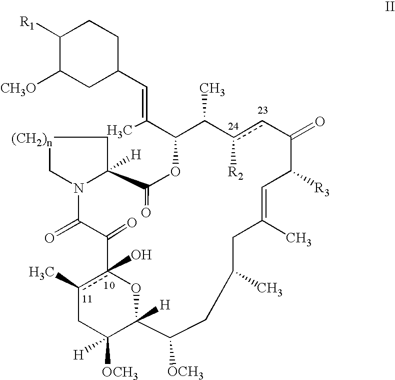
wherein
R₁ is hydroxy or protected hydroxy,
R₂ is hydrogen, hydroxyl or protected hydroxyl,
R₃ is methyl, ethyl, propyl or allyl,
n is an integer of 1 or 2, and
the symbol of a line and dotted line is a single bond.

9. Use of claim 6, wherein the calcineurin inhibitor is a compound of formula 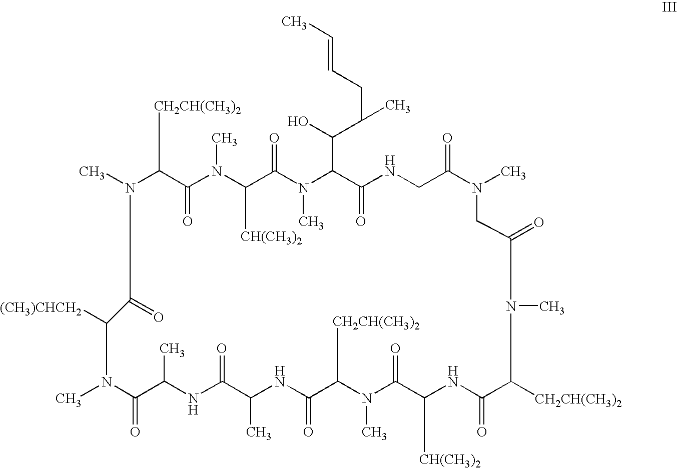
wherein R is methyl, ethyl, propyl, isopropyl or —CH(OH)CH₃.

10. Use of claim 6, wherein the calcineurin inhibitor is a compound of formula Iₚ of claim 2.

11. Use of claim 6, wherein the calcineurin inhibitor is a compound of formula

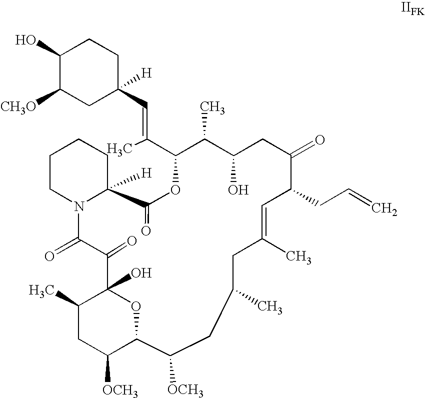

12. Use of claim 6, wherein the calcineurin inhibitor is a compound of formula III wherein R is ethyl.

13. Use of claim 6, wherein the corticoid is selected from the group consisting of hydrocortisone, betamethasone and dexamethasone.

14. Use of claim 6, wherein the calcineurin inhibitor, the corticoid, or both, are in the form of a salt.

15. A pharmaceutical composition comprising, beside pharmaceutically acceptable excipient, in combination a compound of formula Iₚ and hydrocortisone.

16. A pharmaceutical composition comprising, beside pharmaceutically acceptable excipient, in combination a compound of formula Iₚ and betamethasone.

17. A pharmaceutical composition comprising, beside pharmaceutically acceptable excipient, in combination a compound of formula Iₚ and dexamethasone.
